(19) United States
(12) Patent Application Publication
(10) Pub. No.: US 2026/0103666 A1
(43) Pub. Date: Apr. 16, 2026

(54) THREE-DIMENSIONAL CELL CULTURE DEVICE

(71) Applicant: SYNAXYS, Toulouse (FR)

(72) Inventor: Sophie PAUTOT, Toulouse (FR)

(73) Assignee: SYNAXYS, Toulouse (FR)

(21) Appl. No.: 19/114,222

(22) PCT Filed: Sep. 22, 2023
(86) PCT No.: PCT/EP2023/076262

§ 371 Date: Mar. 21, 2025

(30) Foreign Application Priority Data
Sep. 22, 2022 (EP) 22306400.7

(51) Int. Cl. C12M 1/32; C12M 1/12; C12M 1/34; C12M 3/00

(52) U.S. Cl. CPC C12M 23/12; C12M 23/46; C12M 25/00; C12M 41/36; C12M 41/46

(57) ABSTRACT

A cell culture device configured to cultivate cells, the culture device includes a plate with at least one well configured to receive at least one cell to be cultivated, a permeable support sheet, a fixation layer, configured to connect the plate to the permeable support sheet, the fixation layer presenting a predefined shape including at least one wall extending between the permeable support sheet and the plate. The fixation layer is designed in accordance with a pre-defined growing path, in order to model and shape the growth of the cultivated cells outside the well in a controlled and predetermined fashion.

### FIELD OF INVENTION

The present invention relates to three-dimensional (3D) cell culture device.

### BACKGROUND OF INVENTION

Current cell culture devices are designed for 2D adherent cell culture. Their dimensions are defined to enable cell spreading in one single layer: half of the cell is in contact with the surface of the culture device, the other half is bathed by the cell culture medium. The height of these cultures devices is designed to enable a volume of medium suitable for an average of 3 days for an initial seeded density of 20% (surface density).

However, some cell types, and in particular the cells from the nervous system, do not grow well in a 2D culture system. To grow and survive for a reasonable amount of time in vitro meaning at least three weeks, cell types from the nervous system need to be cultivated in 3D culture structures.

The technical problem of 3D cultures is that those culture samples have to extend in the vertical axis (stacking axis). Thus, when traditional 2D culture dishes are used for 3D cultures, the bottom part of the cell culture is further away from the upper surface which is bathed by the medium. This creates a bias in the cell cultures, and in the long term, induces cell death in the bottom layers.

One solution has been to use a “floating” 3D culture (spheroids, organoids, 3D colloidal assembly) to increase cell viability. However, if these “floating” 3D cultures are not tethered to the dish bottom, changing culture medium becomes a delicate task without aspirating the sample. To address this issue, cell culture plates have been designed with cone shaped wells. The plate is centrifuged to pellet “floating” 3D cultures at the bottom and temporally gluing it to the bottom to enable medium change. However, this step can perturb the cell culture. Furthermore, the total volume of these wells is dimensioned for a low cell number and it is not sufficient to maintain high density 3D culture for more than 24 h.

Since 3D samples extend in the vertical stacking axis, to enable observation by microscopy without having to remove the sample from its original container, a thin clear bottom interface is required. Specific cell-culture plates are commercially available with thin clear bottom but these plates are designed for 2D culture and once the sample is placed in the well you cannot get it out without destroying it.

For scientists wishing to study the communication or information propagation between cells or cells culture, it is furthermore essential that said communication is clearly controlled. Cultivated cells or cell cultures have to be put in communication by controlled ways. Such culture connection already exists, for example via medium sharing through semi-permeable membranes; (trans-well strategy, lab-on chip). Medium sharing enables scientists to address questions regarding the influence of secreted factors one the network as whole, but does not provide information regarding communication through physical contact. To address how and at what speed neurons send information to each other scientists and operators have to control the architecture of the neuronal network to know the length of the dendrite and axon involved and thus be able to distribute captor in space to measure the signal propagation.

An alternative method is the physical contact between 3D cell culture by means of microfluidics devices. However, in those cases the cell culture volume is too small for long term culture. Such a small volume is furthermore not compatible with 3D colloidal culture, and it does not allow interfacing of the network with electrodes in 3D for measuring electronical communication, for example. In those microfluidic devices, samples of cell cultures are difficult to recover for further analysis.

There is therefore a need for a 3D cultivation method and device enabling long term survival of the cell culture and enabling a controlled communication between cells or cell cultures, in order to enable proper and precise scientific observations and analysis.

### SUMMARY

The present invention aims at solving this problem and thus relates to a three-dimensional cell culture device configured to cultivate cells in a liquid culture medium, said culture device comprising:
a plate comprising at least one well configured to receive at least one cell to be cultivated,
a permeable support sheet configured to retain the at least one cultivated cell while allowing the liquid culture medium to flow through,
a fixation layer, configured to connect the plate to the permeable support sheet, said fixation layer presenting a predefined shape comprising at least one wall extending between the permeable support sheet and the plate,
   wherein the fixation layer is designed in accordance with a pre-defined growing path, in order to model and shape the growth of the cultivated cells outside the well in a controlled and predetermined fashion.

This way, the solution enables to reach the here-above mentioned objective. Especially, it enables to handle at least one 3D neuronal cell culture in a controlled and organized way, and to control the communication between several cell cultures. More particularly, it allows:
to standardize cell culture dimensions by defining its volume via the dimension of the wells,
to enable the handling of each cell culture without perturbing the cultivated cells,
to facilitate cell harvesting for molecular analysis,
optical imaging of the culture without having to disturb the cultivated cells,
to control physical contact between cells of two different 3D cell culture (neuronal or others),
to control the growth (length, direction, etc.) of specific cell structures, like for example the length of an axon.

The cell culture device according to the invention may comprises one or several of the following features, taken separately from each other or combined with each other:
the cell culture device can further comprise a cultivation scaffold configured to fit inside the well and serve as a growth support for the at least one cultivated cell, thus leading to a three-dimensional cell growth,
the fixation device is applied according to a pattern to define at least one conduct extending from the well along at least a part of the predefined growth path,
said at least one conduct extends transversally to the stacking axis on the support sheet,
said at least one conduct comprises a network of conducts connected to each other,
the support sheet has a meshed structure with meshes having a size preventing the cultivation scaffold to pass through while enabling the liquid culture medium to flow through,
the culture device may comprise at least one signalling system comprising cell relevant signalling molecules, said signalling system being positioned on the permeable support sheet outside the well and being designed according the predefined growth path,
the fixation layer may be designed in accordance with the design of the signaling system in order to model and shape the growth of the cultivated cells outside the well in a controlled and predetermined fashion,
the plate may comprise a second well,
the fixation layer may present a design which comprises at least one wall and one conduct configured to connect the two wells,
the fixation layer may be made of glue,
the plate may present a rectangular shape with a side length of 2 cm,
the plate and the permeable support sheet may be made of Polydimethylsiloxane (PDMS),
the fixation layer and the permeable support sheet may be made of a transparent material in order to enable an observation of the cultivated cells through the cell culture device,
the cell culture device may be configured to cultivate at least one cell emitting electrical signals, the cell culture device being thus configured to be completely immerged in a liquid culture medium, the cell culture device further comprising, inside each well, at least one electrode configured to measure the electrical signals of the at least one cultivated cell,
each well may comprise at least two electrodes configured to communicate with each other,
the cell culture device may further comprise a support piece configured to cooperate with the permeable support sheet in order to enable a safe and easy displacement of the at least one well of the plate without altering of the fixation layer,
the support piece may further enable the plate to be maintained inside the liquid culture medium.

A further object of the present invention relates to a cell culture method carried out by means of a cell culture device according to the technical features listed here-above, wherein said method comprises the steps of:
defining the growth path,
assembling the cell culture device and physically creating the defined growth path within the fixation layer,
immersing said cell culture device inside liquid culture media,
introducing at least one cultivated cell inside the well.

The cell culture method may further comprise adding the signaling system on the permeable support sheet along the growth path.

The cell culture method may further comprise observing and measuring the cell growth.

Regarding this cell culture method, the cell culture device might be connected to a power source configured to feed each electrode and to an external acquisition system configured to collect data measured by said each electrode and the cultivated cell might be an electrical cell emitting electrical signals, the electrical activity of the electrical cell being recorded and measured by means of each electrode.

### BRIEF DESCRIPTION OF THE DRAWINGS

The invention will be better understood, and other aims, details, characteristics and advantages thereof will emerge more clearly on reading the detailed explanatory description which follows, of embodiments of the invention given by way of illustration, purely illustrative and non-limiting examples, with reference to the accompanying drawings:

FIG. 1 is a schematic perspective view of one example of a cultivated cell, a neuron

FIG. 2 is a view from above of a cell culture without the present invention (left) and with the present invention (right),

FIG. 3a is a schematic axial section of a plate comprising two wells according to the present invention,

FIG. 3b is a view from above of a plate comprising four wells according to the present invention,

FIG. 4a is a view from above of a permeable support sheet according to the present invention,

FIG. 4b is a view from above of a particular permeable support sheet according to FIG. 4a, the support sheet being a mesh,

FIG. 5 is a schematic sectional view of the three layers stacked along the stacking axis X according to the present invention,

FIG. 6, is a schematic sectional view of the cell culture device according to the present invention,

FIG. 7 is a view from above of the cell culture device according to the present invention,

FIG. 8 is a schematic perspective view of the cultivation scaffold used for the 3D culture of the cultivated cells inside the wells according to the present invention,

FIG. 9a is a schematic axial section view of the fixation layer according to the present invention,

FIG. 9b is a similar view as the view of FIG. 9a with two connected cultivated cells,

FIG. 10 is a view schematic from above of the fixation layer according to the present invention,

FIG. 11 is a perspective view of the culture device according to the present invention, connected to an electrode,

FIG. 12 js a schematic perspective view of the electrode of FIG. 11 connected inside a well,

FIG. 13 is respectively a schematic from above view and perspective view of a particular embodiment of the present invention presenting one single well,

FIG. 14 is a schematic illustration of a process of emptying a well inside a tube,

FIG. 15 is a schematic axial section of a particular shape of a well according to the present invention,

FIG. 16 is a schematic sectional axial view of another embodiment a cell culture device configured to cooperate with a lab-on-chip device.

### DETAILED DESCRIPTION

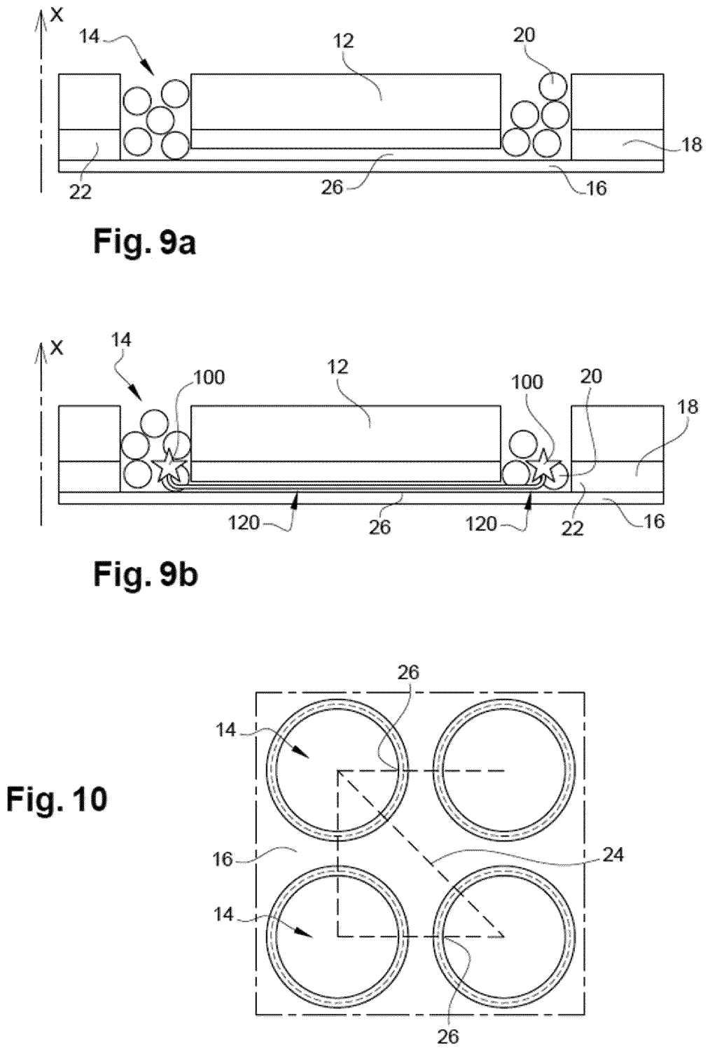

As can be seen on FIGS. 2 and 9, the present invention is about a cell culture device 10 configured to cultivate cells 100. Those cells are part of a culture that might comprise (among other cells and cell types) neurons, progenitor cells, glial cells such as astrocytes and oligodendrocytes, and/or microglial cells.

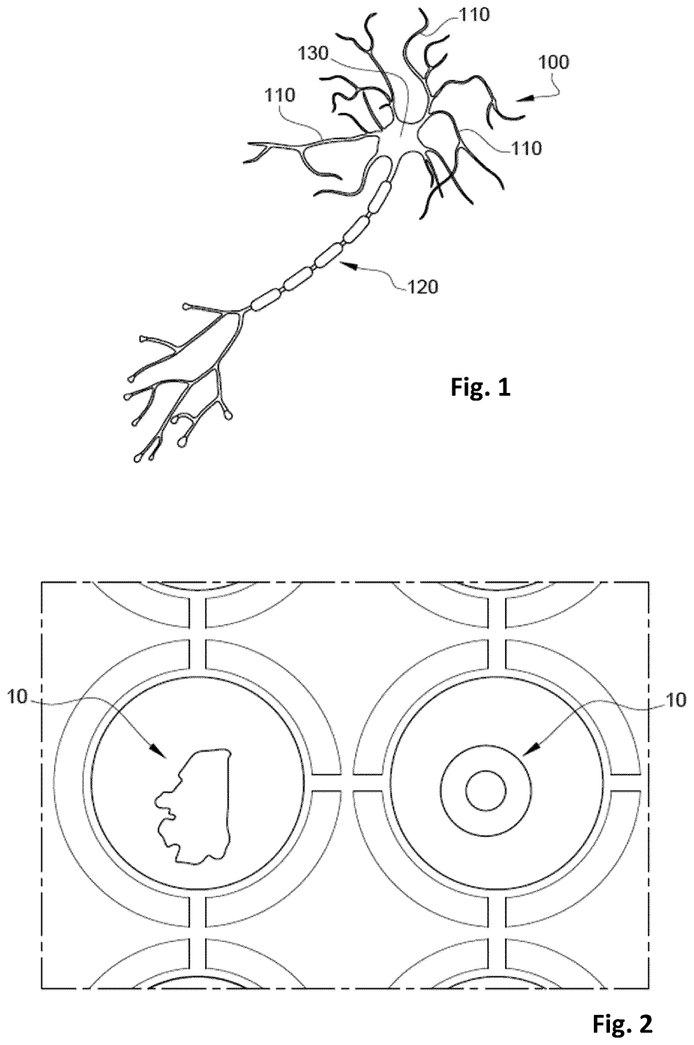

More particularly, the present invention aims at cultivating cells 100 from the nervous system, for example brain cells also called neurons. FIG. 1 depicts an example of cultivated cell 100, a neuron. In the example of the neuron, such a cell 100 displays a particular shape: several dendrites 110 and one axon 120 develop around a central cell body 130 (see FIG. 1) and enable the different neurons to interconnect and communicate with each other by means of electrical and biological messages. Those cells 100 therefore develop by generating a network and connect to each other whenever it is possible. It is therefore important to be able to structure said network construction in order to be able to monitor and understand the precise development mechanisms those cells are involved in. Neurons grow and develop in a viable way if they are surrounded and followed in their development by healthy glial cells. Glial cells maintain homeostasis, oligodendrocytes and Schwann cells form myelin, and provide support and protection for neurons. Glial cells are therefore part of the cultivated cells 100 in the present invention. Their development and growth are equally important than those of the neurons, because neurons cannot properly communicate and/or survive without them.

FIG. 2 shows the difference between a controlled and structured growth environment (on the right) and a chaotic uncontrolled growth environment (on the left). The structure on the left is very difficult to analyze and cannot lead to strong and solid scientific results. More particularly, in scientific research, repeatability of an experiment is fundamental. A controlled and structured growth environment enables standardization, normalization and minimization of the mechanical perturbation and therefore enables repeatability of the scientific experiment.

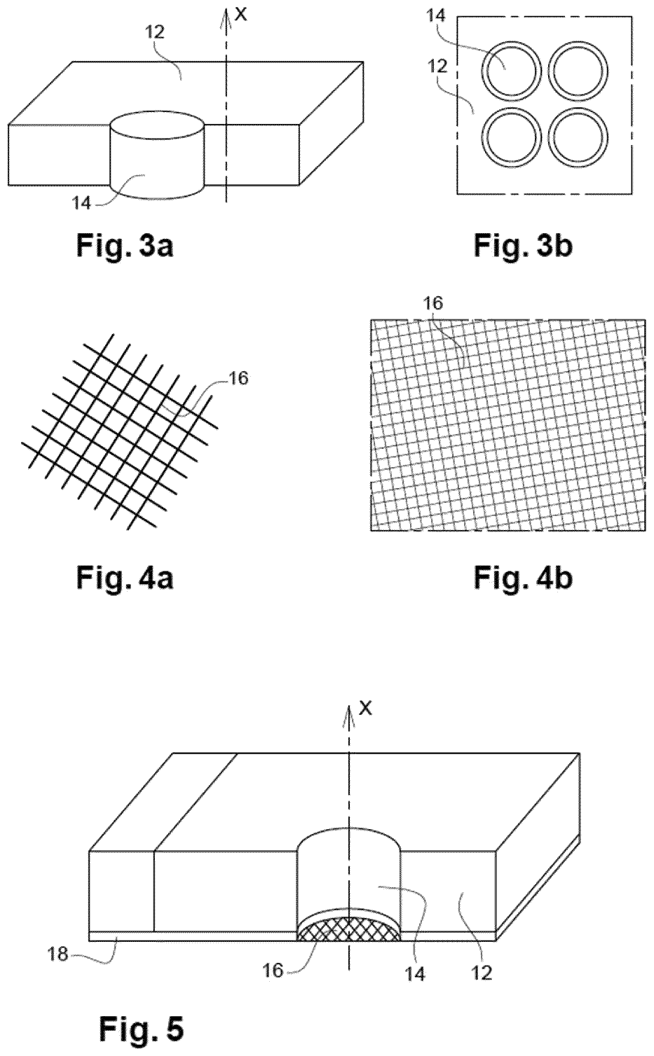

Accordingly, the culture device 10 according to the present invention comprises:
a plate 12 comprising at least one well 14 configured to receive at least one cell 100 to be cultivated (see FIGS. 3a and 3b),
a permeable support sheet 16 (see FIGS. 4a and 4b),
a fixation layer 18 (see FIGS. 5, 9a and 9b).

The plate 12 preferably presents a rectangular shape with a side length of about 2 cm. More generally speaking, the present invention uses the terminology “plate” referring to any element presenting two opposite faces which extends transversely with regards to a stacking axis X of the cell culture device 10. The width of the plate 12 is preferably of less than 2 cm in order to fit in standard culture plates or inside a tube, such as an Eppendorf E tube®. This enables an easy and safe handling of the cell culture device 10 without using too much material. The plate 12 is preferably made of transparent material in order to facilitate cells observation through it. This eases the observation steps, as an operator simply needs to transfer the cell culture device 10 on a magnifier or a microscope to enable a clear observation of the growing and development of the cultivated cells 100. The plate 12 can for example be made of Polydimethylsiloxane. Preferably, the used material for the plate 12 is cell culture compatible and easy to sterilize and to handle and/or mold and/or shape in order to allow an easy and quick production.

As can be seen on FIG. 3a, the plate 12 comprises at least one well 14. According to the present invention, the well 14 extends along the stacking axis X of the cell culture device 10. More particularly, the well 14 extends between two opposite extremities each one opening in one surface of the plate 12. The well 14 configured to receive at least one cell 100 to be cultivated. All cultivated cells from each well 14 are thus cultivated in the same culture medium. In some cases, cultivation conditions might change from one well 14 to another, by adding perturbating elements inside a defined well 14. Such a perturbator can for example be a foreign cell, like a tumorous cell, for example. In order to offer sufficient culture medium to the cultivated cells 100, each well 14 displays a diameter that ranges from 1 mm to 8 mm. The routine standard of the present invention is of 2 mm of diameter. Each well 14 displays a height, along the stacking axis X of maximal 4 mm. The well 14 presents one free opening and one closed opening. The closed opening is preferably located on the bottom surface according to the stacking axis X. The closed opening is closed by the permeable support sheet 16. The well 14 may present a cylindrical or a conical shape.

In case the well 14 presents a conical shape, its diameter increases along the stacking axis X starting from the bottom surface (from the permeable support sheet 16). In such embodiment, the free opening presents a larger diameter than the closed opening. The free opening of the well 14 may also present rounded or beveled edges (see FIG. 15). Regarding the very small dimensions of the well 14, a conical shape and/or rounded or beveled edges to increase the wetting angle between well and culture medium (usually a liquid medium). Thus a surface tension allows the formation of the liquid drop slightly taller than the height of well 14 which enables the maximization of the medium contained by well 14. This effect could be further increased by a surface treatment of the edges. This enables to make sure that all cultivated cells 100 are bathed inside the cultivation fluid. As the cultivated cells 100 form a network and thus a tissue, the size of the formed tissue depends on the size of the wells 14.

One example of medium are saline solutions supplemented with amino acids, vitamins, growth factors and glucose optimized for the cells grown in the cell culture device

In case of neuronal cell culture (and any other cell from the nervous system), it is important to adapt the growing environment. Therefore, the cell culture device 10 comprises a cultivation scaffold 20 configured to fit inside the well 14. This cultivation scaffold 20 extends along the stacking axis X of the cell culture device 10. In the embodiment depicted, the cultivation scaffold 20 is preferably a series of cultivation beads 20 configured to fit inside the well 14. Those cultivation beads 20 are made of glass or gel and typically measure 30 to 100 μm. The cultivation beads 20 are stacked up inside the well 14 along the stacking axis X of the cell culture device 10, as can be seen on FIG. 8. The cultivation beads 20 serve as a growth support for the cultivated cells 100. The well 14 is thus configured to enable and lead to a three-dimensional cell growth. As mentioned above, those three-dimensional growth is important to ensure a reasonable longevity (preferably about several months) of neuronal cells (and any other cell type from the nervous system) which are not keen on growing and developing in two-dimensional environment.

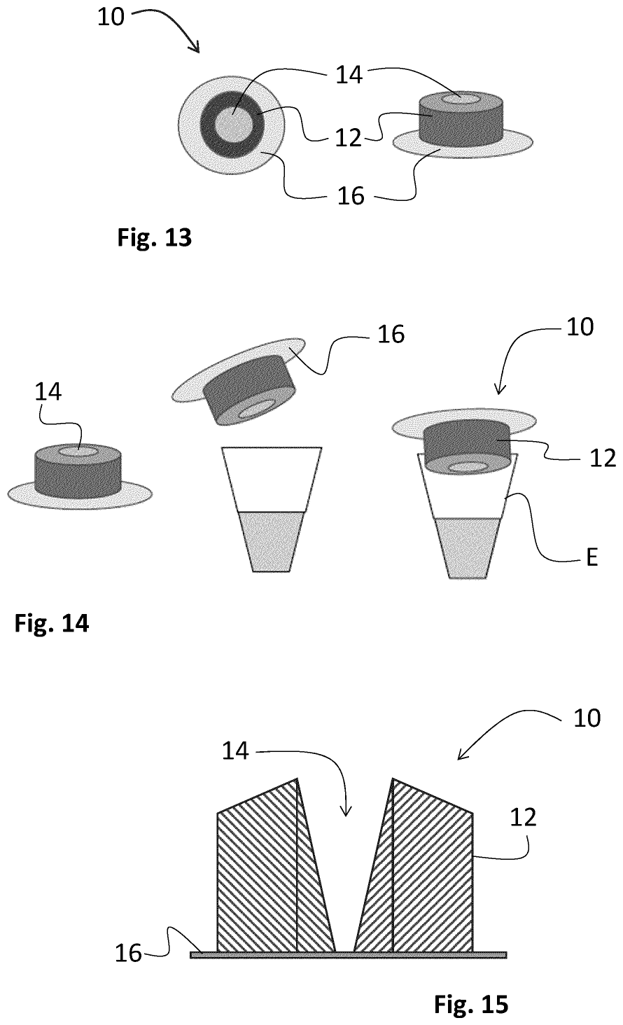

As can be seen on the particular embodiment represented of FIGS. 13 and 14, the cell culture device 10 comprises at least one well 14. This embodiment of the cell culture device 10 is conceived to fit inside a tube, such as an Eppendorf E tube® (see FIG. 14) and thus presents a total diameter ranging from 5 to 8 mm (for accommodating 0,5 to 2,5 mL tubes) and a maximal total height of 4 mm. In order to study and observe the interaction and communication of cultivated cells 100, especially in case of neurons, the plate 12 comprises at least two wells 14. Preferably, one single plate 12 may comprise 1 to 24 wells 14.

As can be seen on FIG. 5, the plate 12 is secured on the permeable support sheet 16 along one of its surfaces, preferably the bottom surface, according to the stacking axis X. The permeable support sheet 16 is configured to retain the cultivated cells 100 while allowing liquid culture medium to flow through. The permeability of the permeable support sheet 16 should be comprised between 10 μm and 100 μm. The permeable support sheet 16 is preferably made of a flexible and/or soft material which can be deformed under a user's pressure. Such a deformation might help to pull any cultivated cell 100 out of the well 14 if needed (see FIG. 14). The support sheet 16 is further preferably configured to enable a retrieval of a sample portion of the support sheet 16 where cultivated cells are localized to be easily separated from a remaining portion of the support sheet 16. The sample portion of the support sheet 16 can be cut, punched or separated in any other suitable manner from the remaining portion. For example, as can be seen in FIGS. 3a and 3b, the permeable support sheet 16 may have a meshed structure (at least partially) with meshes having a size smaller than the size of the cultivation beads 20 (or any other cultivation scaffold 20). The permeable support sheet 16 can for example be a meshed sheet while enabling neurite to let out and the liquid culture medium to circulate through and feed the cultivated cells 100 inside (or outside) the well 14. The cultivated cells together with the sample portion of the support sheet 16 can be collected by means of a biopsy punch.

In order to encourage the cultivated cells 100 to explore the surroundings of the well 14 and orientate their growth outside the well, the support sheet 16 preferably comprises some directional elements, like for example a meshed sheet with threads extending in one single direction.

The fixation layer 18 is configured to connect the plate 12 to the permeable support sheet 16. The fixation layer 18 is further configured to enable the connection of two wells 14 of the plate 12, thus enabling cell 100 communication. The fixation layer 18 is for example made of glue, molded material, or thermofused polymer film. The material should be easy to spread over the support sheet 16 and the plate 12 and easy to be sculpted before drying out and sticking the plate 12 and the permeable support sheet 16 together. The fixation layer 18 is preferably made with liquid glue which is cast around pre-disposed space holders. For example, epoxy glue, silicone glue or liquid PDMS. In an alternative embodiment, the fixation layer is made of UV glue and is made by means of a photo-mask. In this regard, the fixation layer 18 presents, regardless of the used material, a predefined shape comprising at least one wall 22 (see FIGS. 9a and 9b) extending between the permeable support sheet 16 and the plate 12, along the stacking axis X of the cell culture device 10. More particularly, the at least one wall 22 extends between an upper face (with regards to the stacking axis X) of the permeable support sheet 16 and a lower face (with regards to stacking axis X) of the plate 12. The fixation layer 18 is designed in accordance with a pre-defined growing path 24 (see FIG. 10). The fixation layer 18 is built around the designed growing path 24. In some alternative embodiment, this pre-defined growing path 24 is sculpted inside the fixation layer 18. Said growing path 24 is, in both ways, configured to model and shape the growth of the cultivated cells 100 outside the well 14 of the plate 12, along the permeable support sheet 16, in a controlled and predetermined fashion. This way, the length of a dendrite 110 or axon 120 of a cultivated cell 100 (in this case a neuron) can be known and an information propagation speed or a growth speed can easily be calculated. It also enables to decide which wells 14 can be put in contact and which wells 14 should be isolated. Henceforth, as already mentioned, the design of the fixation layer 18 comprises at least one wall 22 extending between the permeable support sheet 16 and the plate 12. The fixation device is hence applied according to a pattern to define one or several conducts delimited by the wall 22 and extending from the well 14, transversally to the stacking axis on the support sheet along at least a part of the predefined growth path. The conducts may be connected to each other to form a network.

Preferably, in order to put two (or more) wells 14 in communication, the fixation layer 18 further comprises at least one conduct 26 extending between said wall 22, the permeable support sheet 16 and the plate 12. The fixation layer 18 may present a height lower than 100 μm. If the fixation layer 18 is too thick, imagery becomes more difficult.

In case the plate 12 comprises two wells 14, the conduct 26 is preferably configured to connect the two wells 14 of the plate 12. In those cases, the conduct 26 extends transversally with regards to the stacking axis X of the cell culture device 10. Such a conduct can present a length comprised between 10 μm and 500 μm. In case the plate 12 comprises more than two wells, the fixation layer 18 might comprise several conducts 26 connecting the different wells 14 together. Those conducts 26 might present different transversal orientations, different lengths, might be interconnected in any relevant fashion regarding the experiment to be carried out. Depending on the design, some wells 14 might be isolated from the others, as to serve as a reference or a control.

In order to enable an easy observation of the cultivated cells 100 through the cell culture device 10, additionally to the plate 12, the fixation layer 18 and the permeable support sheet 16 are all three preferably made of a transparent material. More particularly, it is sufficient for observation purposes if the fixation layer 18 and the permeable support sheet 16 are transparent as those are the element on/through which the cultivated cells 100 evolve and grow.

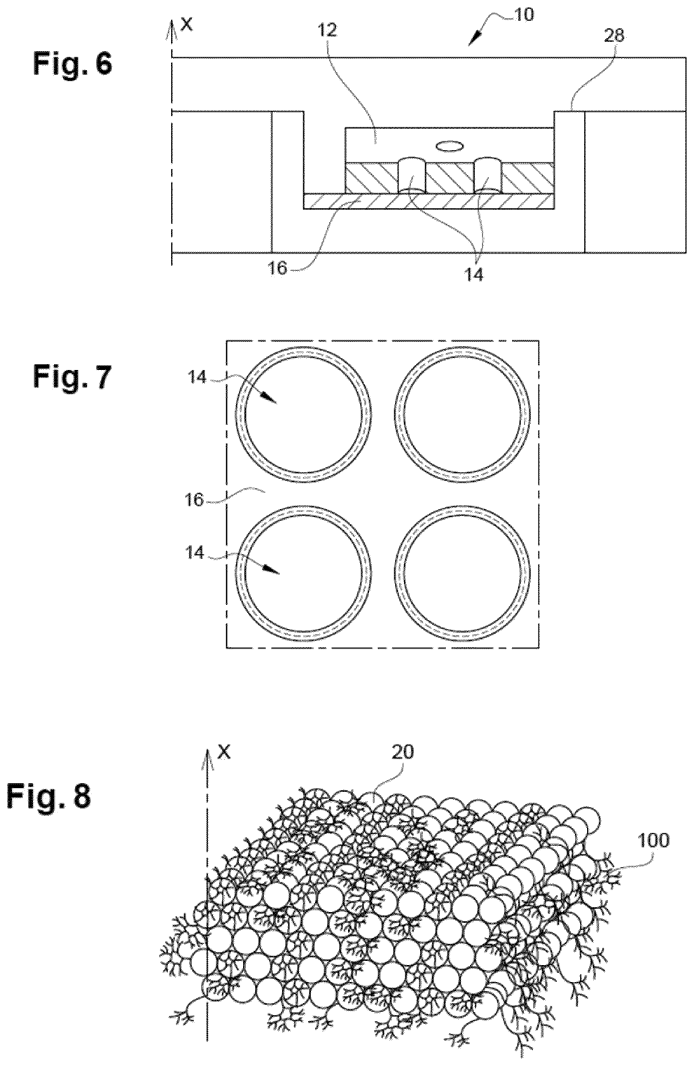

As can be seen on FIG. 6, the cell culture device 10 according to the present invention further comprises a support piece 28 configured to receive one or several cell culture devices 10 in order to enable a safe and easy displacement of the cell culture devices 10 without altering and/or changing the structure of the fixation layer 18.

This support piece 28 further enables the plate 12 to be safely completely immerged inside a culture medium filling a culture dish, without squeezing or stretching the fixation layer 18 and/or damaging the wells 14 and thus the cultivated cells 100 (see FIG. 6). The support piece 28 can be porous or comprise apertures, in order to enable the culture medium to circulate freely. Said support piece 28 further offers the possibility to change cell culture medium at once by lifting the cell culture device 10 up and placing it in a new culture dish containing new culture medium. The support piece 28 may have centering elements configured to center the support piece 28 with respect to the culture dish.

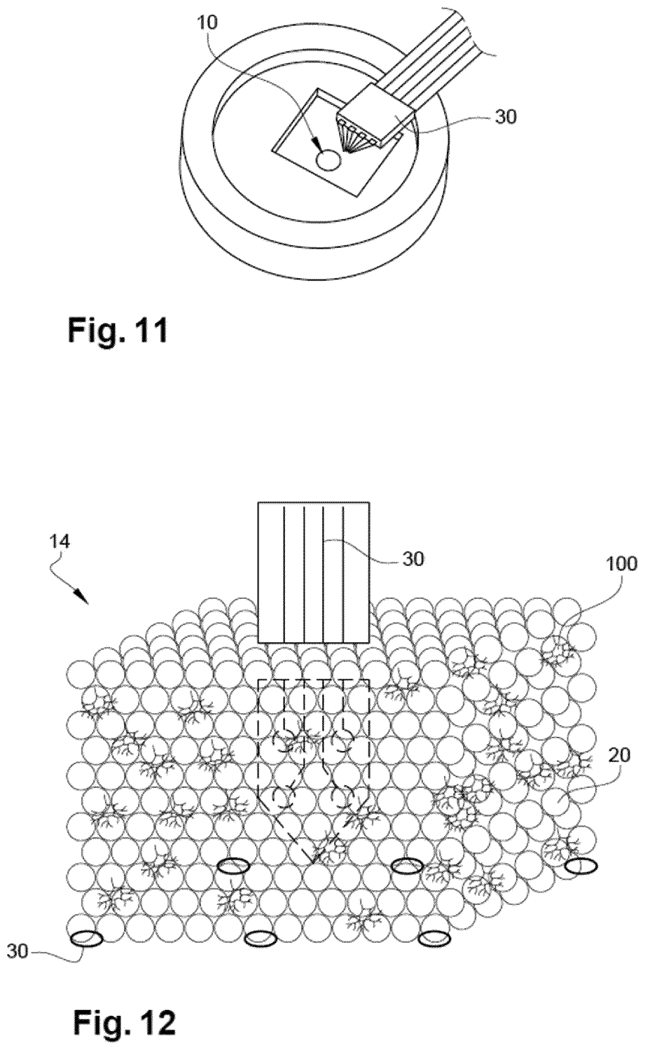

As the plate 12 is completely immerged in the liquid culture medium, the electrical activity of the cultivated cells 100 can be easily monitored by at least one electrode 30 (see FIG. 11). Therefore, as can be seen on FIGS. 11 and 12, the cell culture device 10 may further comprise, inside each well 14, at least one electrode 30.

More particularly, as shown on FIGS. 11 and 12, each well 14 comprises at least two electrodes 30 configured to communicate with each other, thus enabling a precise monitoring of the electrical communication of the cultivated cells 100.

It is well known that neuron cells communicate with each other using neurotransmitters which induce, along the axons 120, an electrical potential modification around the axon 120 membrane and thus generate an electrical signal which spreads along said axon 120 and reaches the next cell 100. This electrical potential modification around the axon 120 membrane induces changes inside the electrical field around the cells 100, inside the well 14, and those changes can be measured by the electrodes 30. As the length of the axons 120 are known thanks to the conducts 26 of the fixation layer 18, one has:
a signal source (cell 100 from first well 14)
a signal destination (cell 100 from second well 14)
a known signal path length (length of the conduct 26 between the two wells 14).
   With all those known parameters, an information propagation speed can thus easily be calculated.

The present invention thus enables a guided growth, allowing to probe directional connectivity and the time it takes to carry an information from A to B.

As already mentioned here-above, the shape and the design of the growing cell network is determined by the shape of the permeable sheet 16 and the shape of the fixation layer 18. However, in order to completely control (and motivate) the growth of the individual cells 100 inside the frame given by the structure of the fixation layer 18, the culture device 10 further comprises at least one signaling system. This signaling system comprises cell relevant signaling molecules as for example cyclic AMP (cAMP) which is known to promote axonal growth of a neuron (see element 120 on FIG. 1). The signaling system is deposited on the permeable support sheet 16 along the conduct(s) 26, outside the wells 14. This signaling system is designed according to a predefined growth path, complementary to the structure of the fixation layer 18. Without this signaling system, the cultivated cells 100 would most certainly stay in the well 14 where the living conditions are suitable. The extra effort to explore their surroundings outside the well to create some connection is induced and motivated by the signaling system.

The signaling system further enables the glial cells mentioned at the beginning of the present specification to develop along the neurons and therefore to ensure their survival and healthy functioning.

The fixation layer 18 is thus designed in accordance with the design of the signaling system in order to model and shape the growth of the cultivated cells 100 outside the well 14, inside the different conduct(s) 26 in a controlled and predetermined fashion.

The cell culture device 10 according to the present invention enables to carry out a cell culture method which comprises the steps of:
defining the growth path 24,
assembling the cell culture device 10 and physically creating the defined growth path 24 within the fixation layer 18,
adding the signaling system on the permeable support sheet 16 along the growth path 24,
immersing said cell culture device 10 inside liquid culture media,
introducing at least one cultivated cell 100 inside the well 14,
observing and measuring the cell growth by any relevant means.

The cultivated cells can be collected together with the sample portion of the support sheet 16 separated from the remaining portion of the support sheet 6, for example by means of a biopsy punch.

In some cases, the cultivated cell 100 is an electrical cell emitting electrical signals, and in order to record and measure the electrical activity of the electrical cell, the cell culture device 10 comprises, as already mentioned, at least one electrode 30 and the cell culture device 10 is connected to a power source configured to feed each electrode 30 and to an external acquisition system configured to collect data measured by each electrode 30.

The described method also offers the possibility of treating the cultivated cells 100 with foreign agent (chemical compound, virus, foreign cells, for example) before measuring their electronic activity.

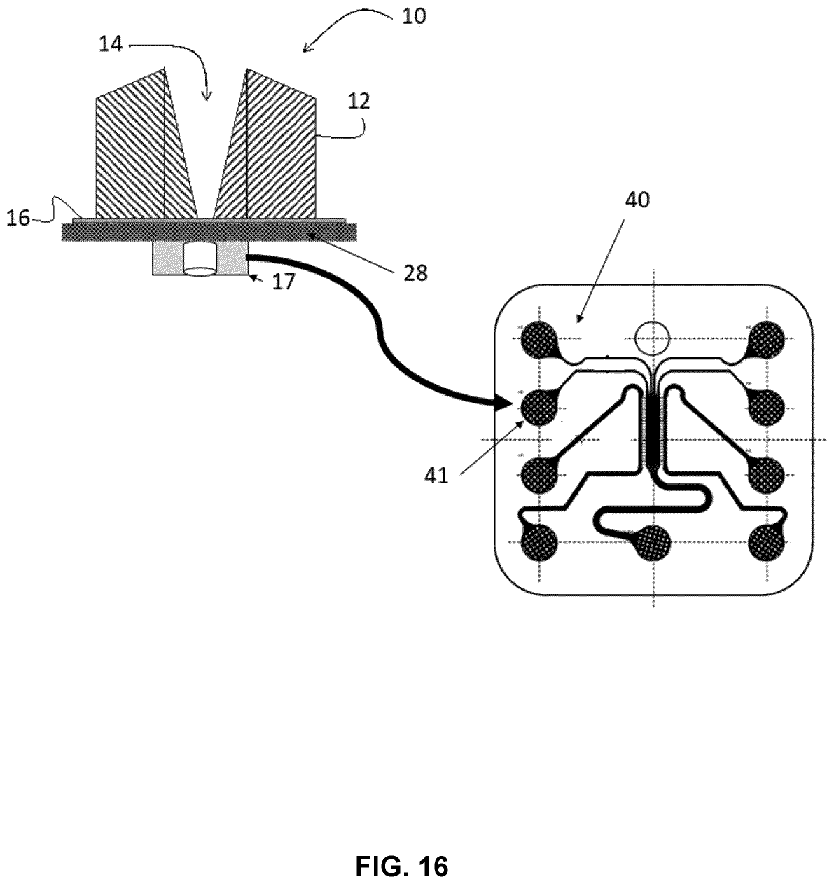

The cell culture device 10 further offers the possibility to put the cultivated cells 100 network in contacts with other cell type, for example by plugging them on an existing lab-on-chip device 40 while being pre-wiring to record the cultivated cells 100 activity. In this respect, FIG. 16 shows a cell culture device 10 having a protrusion 17 extending from a bottom of the support piece 28 along the stacking axis X, opposite the plate 12, and provided with a bore in communication with the well 14. The protrusion 17 is configured to be fitted in an inlet 41 of the lab-on-chip device 40 to enable the cultivated cells in contact with any other suitable tissue type.

1-18. (canceled)

19. A three dimensional cell culture device configured to cultivate cells in a liquid culture medium, said cell culture device comprising:
a plate comprising at least one well configured to receive at least one cell to be cultivated,
a permeable support sheet configured to retain the at least one cultivated cell while allowing the liquid culture medium to flow through,
a fixation layer, configured to connect the plate to the permeable support sheet, said fixation layer presenting a predefined shape comprising at least one wall extending between the permeable support sheet and the plate,
wherein the fixation layer is designed in accordance with a pre-defined growing path, in order to model and shape the growth of the cultivated cells outside the well in a controlled and predetermined fashion.

20. The cell culture device according to claim 19, wherein the cell culture device further comprises a cultivation scaffold configured to fit inside the well and serve as a growth support for the at least one cultivated cell, thus leading to a three-dimensional cell growth.

21. The cell culture device according to claim 20, wherein the support sheet has a meshed structure with meshes having a size preventing the cultivation scaffold to pass through while enabling the liquid culture medium to flow through.

22. The cell culture device according to claim 19, wherein the cell culture device comprises at least one signalling system comprising cell relevant signalling molecules, said signalling system being positioned on the permeable support sheet outside the well and being designed according to the predefined growth path, and wherein the fixation layer is designed in accordance with the design of the signaling system in order to model and shape the growth of the cultivated cells outside the well in a controlled and predetermined fashion.

23. The cell culture device according to claim 19, wherein the plate comprises a second well.

24. The cell culture device according to claim 19, wherein the fixation layer presents a design which comprises at least one wall and one conduct configured to connect the two wells.

25. The cell culture device according to claim 19, wherein the fixation layer is made of glue.

26. The cell culture device according to claim 19, wherein the plate presents a rectangular shape with a side length of 2 cm.

27. The cell culture device according to claim 19, wherein the plate and the permeable support sheet are made of Polydimethylsiloxane.

28. The cell culture device according to claim 19, wherein the fixation layer and the permeable support sheet are made of a transparent material in order to enable an observation of the cultivated cells through the cell culture device.

29. The cell culture device according to claim 19, wherein the cell culture device is configured to cultivate at least one cell emitting electrical signals, the cell culture device being thus configured to be completely immerged in a liquid culture medium, the cell culture device further comprising, inside each well, at least one electrode configured to measure the electrical signals of the at least one cultivated cell.

30. The cell culture device according to claim 29, wherein each well comprises at least two electrodes configured to communicate with each other.

31. The cell culture device according to claim 19, wherein the cell culture device further comprises a support piece configured to cooperate with the permeable support sheet in order to enable a safe and easy displacement of the at least one well of the plate without altering of the fixation layer.

32. The cell culture device according to claim 31, wherein the support piece further enables the plate to be maintained inside the liquid culture medium.

33. A cell culture method carried out by a cell culture device according to claim 19, wherein said method comprises the steps of:
defining the growth path,
assembling the cell culture device and physically creating the defined growth path within the fixation layer,
immersing said cell culture device inside liquid culture media,
introducing at least one cultivated cell inside the well.

34. The cell culture method according to claim 15 when dependent from claim 22, further comprising adding the signaling system on the permeable support sheet along the growth path.

35. The cell culture method according to claim 33, further comprising observing and measuring the cell growth.

36. The cell culture method according to any of claim 33, wherein the cell culture device 10 is connected to a power source configured to feed each electrode and to an external acquisition system configured to collect data measured by said each electrode and wherein the cultivated cell is an electrical sell emitting electrical signals, the electrical activity of the electrical cell being recorded and measured by each electrode.
